(19) United States
(12) Patent Application Publication
(10) Pub. No.: US 2013/0011410 A1
(43) Pub. Date: Jan. 10, 2013

(54) Egg White Antibodies for Prevention and Treatment of Specific Localized Intestinal Infections and Diseases Associated with a Pathogenic Organism or Molecule

(75) Inventors: Hugh B. Fackrell, Windsor (CA); Linton W. Lee, Windsor (CA)

(73) Assignee: Amicus Biotech Inc.

(21) Appl. No.: 13/178,017

(22) Filed: Jul. 7, 2011

(51) Int. Cl. A61K 39/395; A61P 31/04; A61P 31/12

(52) U.S. Cl. 424/157.1

(57) ABSTRACT

The present invention provides the method for prevention and treatment of specific localized intestinal infections and diseases using IgA and IgM antibodies obtained from the eggs of hens which have been hyperimmunized to the same specific infections and diseases. The invention describes the high functionality of IgA and IgM antibodies present in the white of an egg from a hyperimmunized chicken as compared to the IgY from the same egg. The invention also describes the resistance of IgA and IgM to low pH environments such as stomach acids. The invention also describes the inhibition of bacterial growth when bacteria are exposed to IgA antibodies specific to said bacteria together with lysozyme from egg whites.

### STATEMENT REGARDING FEDERALLY SPONSORED RESEARCH OR DEVELOPMENT

N/A

### BACKGROUND OF THE INVENTION

a) Antibodies and Localized Intestinal Infection Critical to intestinal immunity is the production of antibody which prevents gut surface infection. Particularly in mammals, T-cell lymphocytes, found in Peyer's patches, recognize a pathogen's surface proteins which are responsible for binding to the intestinal mucosa. These cells direct B cells to produce antibodies to these binding sites. Said antibodies bind to the pathogen's binding sites. If there is no adhesion of the pathogen to the intestinal mucosa, there can be no infection or disease. Antibodies present in the locale of the gut prevent adhesion of the pathogen to the mucosa, thus preventing infection and disease. Infectious and disease causing pathogens which are bound by antibodies in the intestine are passed on through the gut and excreted.

The required locale for intestinal antibodies to have the most protective function in a mammal is in the upper portion of the small intestine closest to the stomach. Peyer's patches which contain T-lymphocytes are located at the end of the small intestine closer to the large intestine. Bile carries with it a large amount of antibodies to the upper small intestine, but the antibodies are produced in the liver which is remote from the intestinal tract and may not be the appropriate antibodies if an intestinal infection occurs.

Antibodies to pathogens in the intestine need not come from the animal itself. An example of this is the intestinal immunity of newborn mammals despite the fact that a newborn's intestine is not capable of adult immunological behaviour. In this case, the newborn's mother passes on passive immunity in the form of antibodies in high concentrations through colostrum ingested by the newborn as soon as it begins to feed. All the pathogens to which the mother has been exposed, and to which she has produced antibodies are accounted for in the colostrum antibodies. Chickens also pass on antibodies to their chicks for passive immunity. Chicken antibodies are provided by the hen and placed in the egg in the yolk and in the white. In the same manner as mammals, all the pathogens that the hen has been exposed to and to which she has produced antibodies are accounted for in the egg yolk and egg white antibodies (Rose et. al. 1974). Pathogens or molecules known to act locally in the intestine and which are bound by antibodies can be bacteria, viruses, protozoa, fungi or toxins.

b) IgY use against localized intestinal infection IgY from chicken egg yolk has been used in purified forms, semi-purified forms and in complete egg yolk in challenge experiments and field research to protect mammals and fish from localized intestinal infectious diseases. In all cases the hens from which the eggs were obtained had been immunized to the specific antigen used in the experiment. Rabbits have been used in challenge experiments where lethal amounts of E. coli were administered by ilieal loop and protected by orally ingested egg yolk from immunized hens (O'Farrelly, Branton and Wanke, 1992). The egg yolk was administered orally in a bicarbonate solution with successful results. Newborn calves have been used in challenge experiments using lethal doses of corona virus with successful results (Ikemori, et. al, 1997). This particular published article shows that only 1/7^{th} of an egg yolk from an immunized hen is required to protect a calf from a lethal dose of corona virus. These published articles make mention of the possible and desirable use of orally ingested egg yolk antibodies for the prevention and treatment of the causes of localized intestinal infection and disease outside of the research lab and in the context of real world health care.

Further information on the subject of using orally ingested IgY from chicken egg yolk can be found by reading “Avian egg antibodies: basic and potential applications” (Kovacs-Nolan & Mine, 2004) and references contained therein. High resistance to degradation of IgY antibodies by stomach acids has been claimed in some literature, e.g. U.S. Pat. No. 7,727,531, field research experimenters generally agree that IgY is not very resistant to mammalian stomach acid. This means that IgY may not reach the locale of the intestine where it is required to prevent or treat the causes of intestinal disease or infection. Experimenters using IgY from egg yolk either protect the IgY antibodies from stomach acid degradation by some means, such as inclusion in bicarbonate buffer solution to counteract stomach acids, coating IgY granules with commercially available enteric coatings resistant to low pH environments, or use only newborn subjects which naturally have only small amounts of stomach acid. The degradation of the total IgY binding functionality in a given, repeatable, sample at different pH levels has been studied explicitly (Lee, Chang, Lee, Lee and Koo, 2002). The study shows that IgY is stable at pH 5.0, then the IgY functionality drops to 60% of the original level after exposure to pH 4.0. Functionality drops to 30%-40% of original after exposure to pH 3.0; finally a drop to near 0% functionality is evident after exposure to pH 2.0.

c) IgA and IgM in the Literature

Avian IgA and IgM antibodies are known to be present only in the white portion of chicken eggs, just as IgY is known to be present only in the egg yolk. Multiple mentions of egg white IgA and IgM have been made in the literature, but only with regard to their existence as antibodies produced by the avian immune system or to the small amounts present in the white relative to the much more plentiful IgY in the yolk (Rose and Orlans, 1981). Since IgA and IgM in egg white are present in much smaller absolute amounts than IgY in the yolk, only IgY has been explored as a useful product on a commercial scale in either research and clinical diagnostics fields or health care fields.

A discussion of avian immunology shows that immunization with a given immunogen gives rise to the multiple class of antibodies in eggs, IgM, IgA and IgY each specific to said immunogen (Davison, Kaspers and Schat, 2008, p. 277). Class switching shows that the portions of the antibodies, or sites, specific to a given immunogen are exactly the same in all classes of antibodies, IgM, IgA and IgY and are produced by the same B-lymphocytes (Kincade and Cooper, 1971; Martin and Leslie, 1974).

d) Dried Egg Products

Dried eggs are currently used in food products for human consumption and as components of livestock feeds. The most economically efficient method of drying eggs and egg fractions is spray drying. Other methods of drying eggs included hot air circulation across trays (tray drying) and freeze drying or lyophilization. Spray drying eggs should be done at an inlet temperature of 140 degrees Fahrenheit or less in order to preserve the structure of the antibodies contained in the eggs. Spray drying at higher temperatures proportionally reduces the amount of functionality of the antibodies.

### BRIEF SUMMARY OF THE INVENTION

Broadly, the present invention is directed to the use of preparations of egg antibodies from egg whites, known as chicken IgA and IgM, in the prevention and treatment of the causes of localized intestinal infection and disease in fish, birds, human and non-human mammals. IgA and IgM antibodies are obtained from eggs of domestic chickens which have been actively immunized and boosted by direct injection, or other means, against said one or more pathogenic organisms or noxious agents. Immunization is with an immunogen containing immunogenic determinants specific to elicit such antibodies.

Concentration of the antibodies can be done, but it is unnecessary to separate the antibodies from the egg white so that processing and administration are convenient and inexpensive. Antibody produced from egg whites from hens which have been immunized and boosted against specific antigens are effective in locally controlling noxious agents in the intestine, whether viral, bacterial, fungal, protozoal or toxin. The discovery disclosed herein, shows the availability of IgA and IgM antibodies from egg whites in large enough functional quantities to be as, or more, effective as egg yolk antibodies in the control of localized intestinal infection or disease as discussed in the Background. The application of this invention is in the oral administration of IgA and IgM antibodies to fish, birds, humans or non-human mammals to locally control noxious agents in the intestine.

### DETAILED DESCRIPTION OF THE INVENTION

The present invention relates to a new alternative approach, as opposed to antibiotics, for the prevention and treatment of the causes of localized intestinal infection due to bacteria, viruses, protozoa, toxin or fungus.

More precisely, the invention relates to IgA and IgM antibodies from the egg whites of domestic chickens. The IgA and IgM antibodies may be concentrated, but it is unnecessary to separate the antibodies from the egg white, so that processing and administration are convenient and inexpensive.

As used herein, the term “total antibody functionality” refers to the ability of a certain amount of polyclonal chicken egg antibody, of a specific class, to bind to a given antigen. As used herein, the term “antibody prevention” refers to a process by which antibodies (or together with lysozyme) obstruct, delay or destroy intestinal infection or disease causing agents or their products and said antibodies are present in the locale of the intestine prior to the arrival of the infection or disease causing agents.

As used herein, the term “antibody treatment” refers to a process by which antibodies (or together with lysozyme) obstruct, delay or destroy intestinal infection or disease causing agents or their products which are not currently attached to the intestinal mucosa, and said antibodies arrive in the locale of the intestine after the infection or disease causing agents.

IgA and IgM, for the purpose of prevention and treatment of the causes of localized intestinal infection in human and non-human mammals are obtained from eggs of domestic chickens which have been actively immunized and boosted by direct injection or other means against said one or more pathogenic organisms or noxious agents. This invention, i.e. the use of IgA and IgM for the prevention and treatment of the causes of localized intestinal infection in human and non-human mammals, is not obvious for several reasons explained herein.

The invention disclosed herein, includes the discovery that IgA and IgM are obtained in sufficient functional quantities from egg whites to be useful for the purpose of antibody prevention and antibody treatment of the causes of localized intestinal infection or disease. The inventiveness of the method is explained.
1. The total antibody functionality of IgM and IgA together, from an egg white, is the same or greater than the total antibody functionality of IgY in the egg yolk of the same, said egg. Discussion in the Background has illustrated that the total antibody functionality of eggs, specifically in egg yolks, is deemed to be enough for use on a commercial scale in preventing and treating the causes of localized intestinal infection or disease. This said, the amount of IgA and IgM in eggs from immunized hens are of sufficient functionality and quantity to be of commercial scale use through oral ingestion to prevent and treat the causes of localized intestinal infection or disease.
2. IgA and IgM are resistant to mammalian stomach acid, unlike egg yolk antibodies as discussed in the Background. This said, IgA and IgM from immunized hens are not degraded by stomach acid, therefore the egg white antibodies, intact, functional, unprotected by buffering or enteric coating will reach the locale of the intestine after oral ingestion to prevent and treat the causes of localized intestinal infection and disease.
3. IgA from an immunized hen's egg in conjunction with lysozyme naturally found in egg white will inhibit the growth of bacteria to which the IgA antibody is specific through said immunization. This said, IgA contained in egg white in conjunction with lysozyme also found in the same egg white are a natural bactericide.

The techniques for immunization of hens against selected antigens or immunogens are well known to those practiced in the art. The preferred method of immunization is through intramuscular injection of the desired antigen in conjunction with a suitable adjuvant such as Freund's complete or incomplete adjuvant. Booster injections are usually given within a few weeks of the initial immunization. Titers of the desired antibody in the immunized hen's eggs can be confirmed by immunological tests, such as direct ELISA, which are well known to those practiced in the art.

Eggs from immunized hens may be pooled and the antibody extracted by methods explained herein or the egg may be separated and the egg white used in its natural form or dried through spray drying or other means.

### EXAMPLES

The following Examples are given for the purpose of illustration only and are not intended to limit the scope of the present invention.

Each of the following three examples uses eggs from chickens immunized using inactivated E. coli 0011. Immunization through injection with adjuvant was performed on 60 hens using techniques well known to those skilled in the art of immunization. A booster injection was given after approximately 30 days and eggs were collected after approximately 45 days. IgY was obtained from 57 egg yolks and purified through ammonium sulphate precipitation, dialysis and ion exchange. IgA and IgM were obtained from the whites of the same 57 eggs using the method described in U.S. patent application Ser. No. 13/177,114 filed Jul. 6, 2011. Both IgA and IgM were purified through precipitation and ion exchange. The resulting IgY, IgA and IgM titers were in the range of 1:250,000 to 1:500,000 as examined by direct ELISA.

### Example 1

### Agglutination Tests to Show Functionality of IgY, IgA and IgM Antibody Binding Normalized for In-Ovo Volume Proportions

Agglutination titer tests are a measure of the functionality of a particular antibody, not just a quantification of how much antibody is present in the sample. Functionality requires the antibody to bind to the sample antigen in a useful manner causing matrix formation.

Agglutination tests were carried out according to the methods set out by A. A. Benedict in Methods in Immunology and Immunochemistry Vol. 1, p. 229. A two-fold dilution series was performed on each antibody beginning with a 100 μl sample of each of the three antibody types. Samples of suspensions of 10⁷ E. coli 2592 and E. coli 0011 bacteria per treatment well were tested independently. Agglutination titers were read for each E. coli type and each of the three antibodies for all 57 eggs in the study. Volumes of antibody in the whole egg were normalized by multiplying the agglutination titers by the volume of each of the IgY, IgA and IgM containing fractions in in-ovo proportions for each individual egg studied.

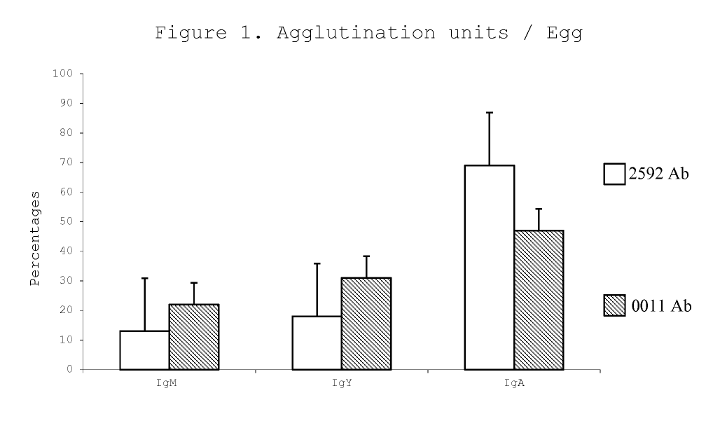

FIG. 1 shows the results of the study of the agglutination units per egg study. The results are presented as a percentage of total, in-ovo, antibody binding functionality possessed by each of the three egg antibody components against each antigen, i.e. IgY, IgA and IgM against E. coli 2592 and E. coli 0011. Using E. coli 2592 antigen the volume normalized antibody functionality measured is IgY: 18%, IgA: 69% and IgM: 13%. Using E. coli 0011 antigen the antibody functionality measured is IgY: 31%, IgA: 47% and IgM: 22%. These results show that the antibody functionality present in in-ovo egg white (i.e. IgA and IgM only) is greater than the antibody functionality present in in-ovo egg yolk (i.e. IgY only).

FIG. 1. description:

FIG. 1. is a histogram and bar graph showing agglutination units measured for IgM, IgY and IgA antibodies to two E. coli antigens expressed as a percentage of total agglutination activity in a single egg. Agglutination to E. coli 2592 shows 13%, 22%, 69%, E. coli 0011 shows 22%, 31%, 47% for IgM, IgY, IgA respectively.

### Example 2

### Functionality of IgA and IgM Antibodies after Exposure to Low pH Environments

IgA and IgM antibody agglutination titers to E. coli 0011 were tested at time 0 and after 1 hour of exposure to a pH 2.0 environment. The pH of the antibody solution was lowered using 0.01M HCl and raised to neutral pH using bicarbonate solution after the prescribed time period. Agglutination tests were carried out according to the methods set out by A. A. Benedict in Methods in Immunology and Immunochemistry Vol. 1, p. 229. A two-fold dilution series was performed on each antibody sample beginning with a 100 μl sample of each of the antibodies. A sample suspension of 10⁷ E. coli 0011 bacteria was used per treatment well. Agglutination titers were read for the two antibodies for all 57 eggs in the study.

IgA at time 0 had a titer of 1:200, after 1 hour the titer was 1:275

IgM at time 0 had a titer of 1:8, after 1 hour the titer was 1:4 All agglutination titers from these two-fold dilution series have no more than a single dilution step difference in titer indicating that there is no significant difference in titer from time 0 to time 1 hour. These results demonstrate the resistance of both IgA and IgM to low pH environments.

### Example 3

### Demonstration of the Ability of the Combination of IgA Antibodies and Lysozyme to Inhibit Bacterial Growth

10⁶ E. coli 0011 bacteria were added to 10 ml nutrient broth for each treatment combination. 1 mg/ml samples of each antibody (IgY, IgA) were used for treatment. A 1 mg/ml quantity of dialyzed, lyophilized lysozyme from chicken egg white obtained from Sigma-Aldrich was used in each appropriate treatment. Treatment Combinations (all treatments in nutrient broth):
1. 10⁶ E. coli 0011
2. Lysozyme+10⁶ E. coli 0011
3. IgA+10⁶ E. coli 0011
4. IgY+10⁶ E. coli 0011
5. IgA+lysozyme+10⁶ E. coli 0011
6. IgY+lysozyme+10⁶ E. coli 0011

E. coli were allowed to enter the log phase for one study and allowed to enter the stationary phase for the second study. Counts of Colony forming units (CFU) were taken for each treatment combination at time 0 and after 3 hours incubation at 37° C. for the log phase study and for the stationary phase study. In addition, E. coli samples from the IgA+lysozyme treatments were dispersed and re-suspended in new nutrient broth after the initial study in order to test whether the treatment actually affected the nutrient broth and not the bacteria themselves. A second, independent, experiment measured turbidity, indicating bacteria growth. The treatments consisted of untreated 10⁵ E. coli 0011 per ml in nutrient broth as the control and 10⁵ E. coli 0011 per ml+1 mg/ml IgA+1 mg/ml lysozyme in nutrient broth for each of the 57 eggs in the study. Turbidity was measured in a time step of 15 minutes beginning in the lag phase and through log and stationary phases of growth.

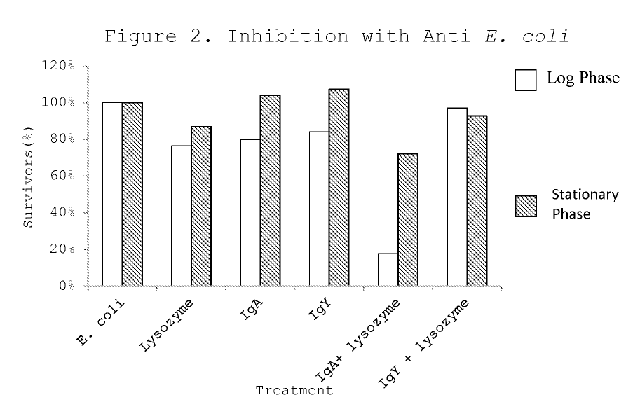

FIG. 2 shows the percent survivors during the log and stationary phases of bacteria growth for each of the treatments. During the log phase of growth the IgA+lysozyme treatment significantly reduced the number of surviving CFU (<20% surviving) after incubation as compared to time 0. All other treatments did not show significant reductions in survivors during the log phase. In the stationary phase the IgA+lysozyme treatment had the greatest reduction in surviving CFU (70% surviving). Re-suspension of E. coli samples from the IgA+lysozyme treatments in new nutrient broth showed no further increase in colony forming units.

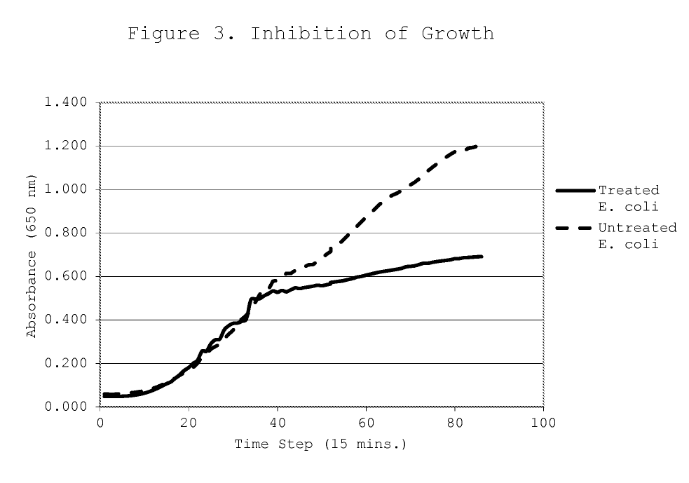

FIG. 3 shows the turbidity measured at 650 nm at a time step of 15 minutes. At approximately time step 38 the E. coli samples treated with IgA and lysozyme showed markedly less turbidity than the control samples of untreated E. coli.

FIG. 2. description:

FIG. 2. is bar graph and histogram showing the survivorship of E. coli colonies in two sets of six treatment groups expressed as a percentage of the untreated E. coli group. The first set of measurements is for treatments beginning in Log phase, the second set is for treatments beginning in Stationary phase. Log phase shows 100%, 76%, 80%, 84%, 18%, 97%, Stationary phase shows 100%, 87%, 104%, 107%, 72%, 93% for untreated, Lysozyme, IgA, IgY, IgA+lysozyme, IgY+lysozyme treatment groups respectively.

FIG. 3. description:

FIG. 3. is a line graph showing growth of treated and untreated E. coli, as measured by absorbance at 650 nm in 15 minute time steps from 0 to 86 steps. Treated and untreated E. coli growth is equal in both groups from step 0 and absorbance of 0.048 until step 34 and absorbance of 0.480. After step 35 the untreated group continues growth to 1.178 absorbance. After step 35 the treated group continues to 0.691 nm absorbance.

Results, shown in all studies in example 3, demonstrate the ability of the combination of IgA and lysozyme to inhibit growth of bacteria, especially during the log phase of growth.

### REFERENCES

U.S. Pat. No. 7,727,531
U.S. application Ser. No. 13/177,114 (filed Jul. 6, 2011)
Benedict, A. A. (1967), Methods in Immunology and Immunochemistry. Vol. 1, p. 229
Davison, F., Kaspers, B, & Schat, K. A. (2008). Avian Immunology. Academic Press
Ikemori, Y., Ohta, M., Umeda, K., Icatlo, F. C. Jr., Kuroki, M., Yokoyama, H., and Kodama, Y. (1997) Passive Protection of Neonatal Calves against Bovine Coronavirus-induced Diarrhea by Administration of Egg Yolk or Colostrum Antibody Powder. Vet. Microbiol., 58, 105
Kincade, P. W. and Cooper, M. D. (1971). Development and Distribution of Immunoglobulin-Containing Cells in the Chicken. An immunofluorescent Analysis Using Purified Antibodies to Mu, Gamma and Light chains. J. Immunol. 106, 371-382
Kovacs-Nolan, J., and Mine, Y. (2004) Avian Egg Antibodies: Basic and Potential Applications. Avian and Poultry Biology Reviews 15 (1), 25-46
Lee, K. A., Chang, s. K., Lee, Y. J., Lee, J. H. and Koo, N. S. (2002) Acid Stability of Anti-Helicobacter pyroli IgY in Aqueous Polyol Solution. Journal of Biochemistry and Molecular Biology, 32(5), 488-493

Martin, L. N. and Leslie, G. A. (1974) IgM-forming Cells as the Immediate Precursor of IgA-Producing Cells During Ontogeny of the Immunoglobulin-producing System of the Chicken. J. Immunol. 113, 120-126
O'Farrelly, C., Branton, D. and Wanke, C. A. (1992) Oral Ingestion of Egg Yolk Immunoglobulin from Hens Immunized with an Enterotoxigenic Escherichia coli Strain Prevents Diarrhea in Rabbits Challenged with the Same Strain. Infect. Immun., 60, 2593
Rose, M. E. and Orlans, E. (1981). Immunoglobulins in the Egg, Embryo and Young Chick. Dev. Comp. Immunol. 5, 15-20

We claim:

1. A method for prevention and treatment of localized intestinal infection and disease associated with a pathogenic organism or molecule in a fish, bird or mammal, said method comprising orally administering to said fish, bird or mammal IgA and IgM antibodies obtained from the egg of a domestic fowl hen which has been actively immunized against said pathogenic organisms or molecules by injection of the hen with an immunogen containing immunogenic determinants specific to elicit said antibodies.

2. The method of claim 1, wherein said pathogenic organism is a virus selected from the group consisting of rotavirus, coronavirus, Norwalk virus, and other enteropathogenic viruses which are causes of localized intestinal infection and disease.

3. The method of claim 1, wherein said pathogenic organism is a bacteria selected from the group consisting of E. coli, S. aureus, H. pylori, C. difficile, V. cholera, Salmonella spp and other enteropathogenic bacteria which are causes of localized intestinal infection and disease.

4. The method of claim 1 wherein said pathogenic organism is a protozoan selected from the group consisting of Giardia, and other enteropathogenic protozoa which are causes of localized intestinal infection and disease.

5. The method of claim 1 wherein said pathogenic organism is a fungus is selected from the group consisting of Candida albicans, and other invading fungi which are causes of localized intestinal infection and disease.

6. The method of claim 1, wherein said molecule is a toxin selected from the group consisting of E. coli toxins, cholera toxins, S. aureus toxins and other enteropathogenic toxins which are causes of localized intestinal infection and disease.

7. The method of claim 1, wherein the immunization of said hen is performed by administration of the antigen in conjunction with a water-in-oil emulsion adjuvant, or other means.

8. The method of claim 1, wherein said fowl is a chicken.

9. The method of claim 1, wherein total functionality of IgA plus IgM is greater than or equal to the total functionality of IgY in the same egg as measured by agglutination titers and then normalized for volumes.

10. The method of claim 1, wherein IgA and IgM antibodies are resistant to acidic environments as low as pH 2.0.

11. The method of claim 1, wherein IgA combined with egg white lysozyme inhibits the growth of bacteria.

12. The method of claim 1, wherein said IgA and IgM antibodies are obtained from the white of said egg.

13. The method of claim 12, wherein the white of said egg is separated into IgA and IgM containing fractions.

14. The method of claim 12, wherein said IgA and IgM is obtained by separating the white of said egg and drying the IgA and IgM containing fractions to form a powder.

15. The method of claim 12, wherein said IgA and IgM is obtained by drying the unseparated white of said egg to form a powder.

16. The method of claim 12, wherein IgA and IgM antibodies are in unseparated, undried white of said egg.

17. The method of claim 12, wherein said IgA and IgM is obtained by drying the unseparated yolk and white of said egg to form a powder.

18. The method of claim 12, wherein IgA and IgM antibodies are in unseparated, undried yolk and white of said egg.
